(19) United States
(12) Patent Application Publication
(10) Pub. No.: US 2008/0146878 A1
(43) Pub. Date: Jun. 19, 2008

(54) Magnification attachment / accessory for laryngoscopes

(76) Inventors: Karen Naimoli Frost, Littleton, CO (US); Raymond Anthony Sirianne, Evergreen, CO (US)

Correspondence Address: BARBER LEGAL, P.O. BOX 16220, GOLDEN, CO, 80402-6004, US

(21) Appl. No.: 11/638,879

(22) Filed: Dec. 14, 2006

(51) Int. Cl. A61B 1/267

(52) U.S. Cl. 600/188

(57) ABSTRACT

The present invention teaches an accessory to a laryngoscope, which may be retrofitted thereto or integrated on a new laryngoscope as original equipment, having at least one fastener allowing attachment to the barrel of a standard laryngoscope, a rail secured to the barrel of the laryngoscope by the fastener, and a lens slide seated on the rail and traveling on thereon parallel to the barrel. On the lens slide, a lens frame mounts a progressive magnification lens, that is, a single optical element lens having multiple magnification powers when viewed through different portions (such as a bi-focal lens). A lens pivot mounted on the lens slide holds the lens frame, so that the lens may be adjusted in angle (and thus distance in the direction perpendicular to the rail).

### CROSS-REFERENCE TO RELATED APPLICATIONS

The application is a continuation-in-part of copending U.S. Provisional Patent Application No. 60/749,949 filed Dec. 13, 2005 in the name of the same inventors, and entitled MAGNIFICATION ATTACHMENT OR ACCESSORY FOR LARYNGOSCOPES, and claims the priority and benefit of that earlier application, the entire disclosure of which is incorporated herein by this reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### FIELD OF THE INVENTION

This invention relates generally to laryngoscope medical devices, and specifically to accessory attachments and retrofit devices for laryngoscopes.

### STATEMENT REGARDING FEDERALLY FUNDED RESEARCH

This invention was not made under contract with an agency of the US Government, nor by any agency of the US Government.

### BACKGROUND OF THE INVENTION

The laryngoscope is a device used by medical personnel to examine or even manipulate a patient's tongue, epiglottis, throat, lips, jaw, and so on during procedures such as incubation of airways. The laryngoscope generally consists of a metal handle of a generally cylindrical configuration having a hollow battery receiver therein, along with circuitry to power a light bulb placed on an elongated blade portion which is inserted into the patient's throat for the actual procedure. The blade portion of the laryngoscope is generally flat or curved, often with a gentle valley on the top surface allowing easier visibility and insertion of tubes therealong.

Laryngoscopes are used as aids to medical practitioners in intubation procedures wherein an endotracheal (ET) tube is inserted into the airway of a patient. The ET tube is used to maintain the patency of the airway and may be attached to a mechanical ventilator to provide oxygenation and/or ventilation. In most cases, intubation is a part of an emergency resuscitation or life-support procedure in which a patient is placed on a ventilator. The breathing tube must be inserted swiftly into the oral cavity, past the glottis, between and past the vocal folds/vocal cords, and lodge in the region distal to the vocal folds and proximal to the carina. Any delay in the intubation process can potentially be detrimental to patient outcome. The laryngoscope has features that aid the practitioner in quickly displacing the patient's tongue caudally (towards the patient's feet) to make visible the glottis and vocal folds. It is critical that the ET tube be inserted gently and quickly between the vocal folds to avoid injury to the airway and surrounding structures anatomy and to correctly position the extreme end of the ET tube (that end distal the practitioner and proximal the lungs). It is therefore of the greatest importance that these anatomical structures be easily identified and clearly visible to the practitioner performing the procedure.

In smaller patients, especially premature infants and underweight infants, as well as in full term neonates, small children, and some very small adults, the vocal folds lie close to the near visual field of the practitioner such that even with conventional reading spectacles, some practitioners with presbyopia cannot easily focus on these anatomical structures. The inability to quickly and clearly gain a view of these critical anatomical structures can prolong the intubation process and delay initiation of life support, thus placing the patient at increased risk of compromised outcome, injury, or possible death.

It will be appreciated that while the laryngoscope is an important aid in viewing the glottis, vocal cords, epiglottis, and related portions of the throat, manipulation of the epiglottis, while important to allow a view of the glottis, is not the end of the exercise: the ET tube must be inserted in correct placement into the patient's airway. The tube must pass through the vocal cords and still have the indicia of length clearly visible to show the placement in terms of distance down the airway in order to avoid placement of the end of the ET tube distal the practitioner to a location past the carina.

Various items may be found in the collection of the United States Patent Office.

U.S. Pat. No. 4,056,311, for “Progressive power ophthalmic lens having a plurality of viewing zones with non-discontinuous variations therebetween” is one such, as are U.S. Pat. No. 4,901,708, for “Viewing Laryngoscope”, U.S. Pat. No. 5,425,356 for “Telescopic Laryngoscope Blade”, U.S. Pat. No. 5,443,058 for “Blade for Telescopic Laryngoscope”, U.S. Pat. No. 5,873,818, entitled “Laryngoscope with Enhanced Viewing Capability”, U.S. Pat. No. 6,356,373 for “Progressive Power Lens and Mold for Producing Same”, and U.S. Pat. No. 6,843,769 for “Optical Luminous Laryngoscope” are examples. In general, it is known to use 18 viewing equipment on laryngoscopes, optical aids for example. Most practitioners may be trained on and thus become accustomed to a single type of laryngoscope (just as they may be trained on and become accustomed to a particular type or style of stethoscope and prefer that type throughout their medical careers) and may not be as facile with other types. However, while the practitioner's laryngoscope may stay the same the practitioner's eyesight will slowly degrade as the practitioner ages, and worse, the structures of interest to the practitioner will normally be closer to the practitioner than normal reading distance, so even reading glasses may not completely make up for the slow loss of visual acuity in the very near visual field, i.e. within approximately 4 to 6 centimeters or less from the practitioner's eye. (Such short distances of work may occur during ET tube insertion.) In addition, this facility with one type of laryngoscope may serve as an obstacle to introduction of new laryngoscopes. It is also worth mentioning that practitioners may find themselves suddenly acting with lost, forgotten, or broken spectacles. In any such situation, it would be beneficial to increase the visual acuity of the practitioner's near visual field in a quick and convenient manner.

In general, it would be advantageous to provide some device allowing easier viewing using a laryngoscope.

It would also be advantageous to provide an attachment which may be fitted to the practitioner's laryngoscope of choice, thus allowing the improved near-field vision without requiring the practitioner to re-learn or alter his or her preferred intubation techniques.

It would further be advantageous to provide an attachment allowing a user to adjust magnification to their vision, and to adjust it quickly and easily, without excessive manipulation during speedy and time critical procedures.

It would further be advantageous to provide a device allowing a user to adjust the focus of the magnification to accommodate the distance to the anatomical structures to be viewed and the distance from the user's eye.

It would further be advantageous to provide a device not requiring complex multi-element lenses, not requiring mechanically focused lens trains, and not requiring a large number of moving parts.

### SUMMARY OF THE INVENTION

General Summary

The present invention teaches an accessory to a laryngoscope, which may be retrofitted thereto or integrated on a new laryngoscope as original equipment, having at least one fastener allowing attachment to the barrel of a standard laryngoscope, a rail secured to the barrel of the laryngoscope by the fastener, and a lens slide seated on the rail and traveling thereon parallel to the barrel. On the lens slide, a lens frame mounts a progressive addition or magnification lens, that is, a single optical element lens having multiple magnification powers when viewed through different portions (such as a bi-focal lens). A lens pivot mounted on the lens slide holds the lens frame, so that the lens may be adjusted in angle (and thus distance in the direction along the axis of the curved or straight laryngoscope blade). In embodiments, the lens pivot may be omitted, the rail and slide may be omitted, additional light may be provided, indicia may be provided on the lens, the frame, the slide or the rail, the fastener may be one or more clamps, clasps, straps or the like.

Thus, the present invention provides a device allowing easier viewing using a laryngoscope, in a device which may be retrofitted to model of laryngoscope already in use, thus allowing the installed base of laryngoscopes to be upgraded, and provides a device allowing a user to adjust magnification to their vision, and to adjust it quickly and easily, without excessive manipulation during speedy and critical procedures, and further allowing a user to adjust the focus of the magnification to accommodate the distance to the object to be viewed and the distance from the user's eye, and yet further not requiring complex multi-element lenses, not requiring mechanically focused lens trains, and not requiring a large number of moving parts.

Summary in Reference to Claims

It is therefore another aspect, advantage, objective and embodiment of the invention, in addition to those discussed previously, to provide a laryngoscope accessory for use with a laryngoscope having a barrel having a circumference, the accessory comprising:
an attachment dimensioned and configured to engage such barrel of such laryngoscope, and:
a progressive lens secured to the attachment, the progressive lens having at least first and second different optical power ratings.

It is therefore another aspect, advantage, objective and embodiment of the invention to provide a laryngoscope accessory wherein the attachment further comprises:
at least one circumferential member passing about such circumference of such barrel, and:
at least one fastener secured to the circumferential member so as to force the circumferential into secure physical engagement with such barrel.

It is therefore another aspect, advantage, objective and embodiment of the invention to provide a laryngoscope accessory wherein the circumferential member further comprises:
a band.

It is therefore another aspect, advantage, objective and embodiment of the invention to provide a laryngoscope accessory wherein the circumferential member further comprises:
a strap.

It is therefore another aspect, advantage, objective and embodiment of the invention to provide a laryngoscope accessory wherein the progressive lens further comprises:
a graduated progressive lens having no discontinuities of optical power.

It is therefore another aspect, advantage, objective and embodiment of the invention to provide a laryngoscope accessory wherein the progressive lens further comprises:
a bifocal lens.

It is therefore another aspect, advantage, objective and embodiment of the invention to provide a laryngoscope accessory wherein the progressive lens further comprises:
a multi-focal step-wise lens.

It is therefore another aspect, advantage, objective and embodiment of the invention to provide a laryngoscope accessory wherein the progressive lens further comprises:
a lens frame secured to at least one portion of the progressive lens.

It is therefore another aspect, advantage, objective and embodiment of the invention to provide a laryngoscope accessory wherein the lens frame further comprises:
a pivot allowing the angle of the progressive lens to be adjusted relative to such blade of such laryngoscope.

It is therefore another aspect, advantage, objective and embodiment of the invention to provide a laryngoscope accessory wherein the attachment further comprises:
a rail affixed to the attachment and thereby fixed relative to such laryngoscope, and:
a slide movably disposed upon the rail, the progressive lens movably attached to the attachment via the slide, whereby:
the progressive lens may be moved parallel to such axis of such barrel of such laryngoscope.

It is therefore another aspect, advantage, objective and embodiment of the invention to provide a laryngoscope accessory wherein the fastener further comprises one member selected from the group consisting of: a clamp, a clip, a snap, a buckle, a thumb-screw, hook-and-loop fabric, elastic and combinations thereof.

It is therefore another aspect, advantage, objective and embodiment of the invention to provide a laryngoscope comprising:
a cylindrical barrel;
a blade attached to a first end of the barrel and extending, curved, therefrom;
a progressive lens secured to the barrel near the first end, the progressive lens having at least first and second different optical power ratings.

It is therefore another aspect, advantage, objective and embodiment of the invention to provide a laryngoscope wherein the progressive lens further comprises:
a graduated progressive lens having no discontinuities of optical power.

It is therefore another aspect, advantage, objective and embodiment of the invention to provide a laryngoscope wherein the progressive lens further comprises:
a bifocal lens.

It is therefore another aspect, advantage, objective and embodiment of the invention to provide a laryngoscope wherein the progressive lens further comprises:
a multi-focal step-wise lens.

It is therefore another aspect, advantage, objective and embodiment of the invention to provide a laryngoscope, wherein the progressive lens further comprises:
a lens frame secured to at least one portion of the progressive lens.

It is therefore another aspect, advantage, objective and embodiment of the invention to provide a laryngoscope wherein the lens frame further comprises:
a pivot allowing the angle of the progressive lens to be adjusted relative to the blade of the laryngoscope.

It is therefore another aspect, advantage, objective and embodiment of the invention to provide a laryngoscope further comprising:
a rail affixed to the barrel, and:
a slide movably disposed upon the rail, the progressive lens movably attached to the laryngoscope via the slide, whereby:
the progressive lens may be moved parallel to such axis of the barrel of the laryngoscope.

### BRIEF DESCRIPTION OF THE DRAWINGS

FIG. 1 is an isometric view of the preferred embodiment of the invention.

FIG. 2 is an exploded view of the preferred embodiment of the invention.

FIG. 3 is an isometric view of a typical curved-blade laryngoscope.

FIG. 4 is an isometric view of a typical straight-blade laryngoscope.

FIG. 5 is an isometric side view showing the details of the barrel clamp components.

FIG. 6 and FIG. 6B are isometric demonstration diagrams showing articulation of the lens of the preferred embodiment of the invention.

FIG. 7 and FIG. 7B are isometric side views which demonstrate longitudinal position adjustment of the lens guide.

FIG. 8 is an isometric view of the preferred embodiment of the invention with the lens frame in the folded or parked position.

FIG. 9 is an isometric view of an alternative embodiment of the invention.

FIG. 10 is an isometric view of an alternative embodiment of the invention attached to a straight-blade laryngoscope.

FIG. 11 is an isometric view of an alternative embodiment of the invention integral to a straight-blade laryngoscope.

FIG. 12 is a cross-sectional view of a simple progressive lens having at least two different optical powers at different locations of the lens.

### INDEX OF THE REFERENCE NUMERALS

20—elastic tensioner of the barrel clamp
21—stationary clasp of the barrel clamp
22—moveable clasp of the barrel clamp
23—lens rail
24—lens slide
25—pivot link
26—pivot fasteners
27—lens frame
28—progressive-magnification lens
29—barrel clamp
31—laryngoscope curved blade
32—laryngoscope barrel
41—laryngoscope straight blade
61—first direction of rotation
62—second direction of rotation
71—first direction of translation (linear motion)
72—second direction of translation (linear motion)
90—integrated lens frame and lens slide
91—thumbscrew
100—progressive lens
102—first light ray
104—second light ray
111—integral barrel and rail

### DETAILED DESCRIPTION

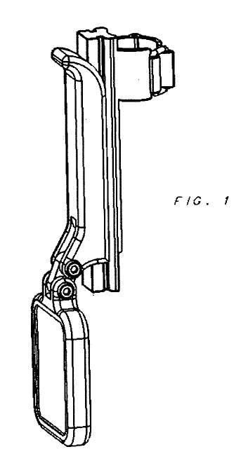

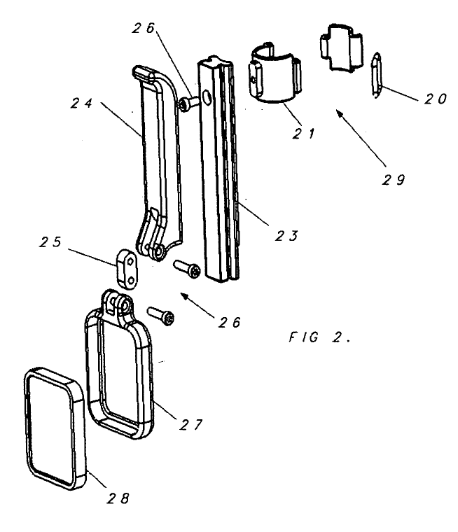

FIG. 1 is an isometric view of the preferred embodiment of the invention, while FIG. 2 is an exploded view of the preferred embodiment of the invention, showing the parts individually.

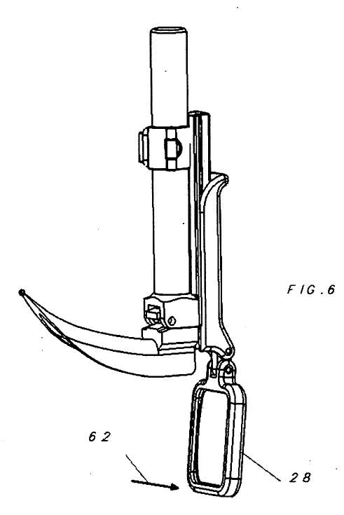

Attachment of the device to the barrel of the laryngoscope is achieved by means of barrel clamp 29, the attachment structure of the preferred embodiment. Elastic tensioner of the barrel clamp 20 may literally be an elastic band, an O-ring or the like, and may hold the two clasps of the clamp in tight physical engagement with the barrel of a laryngoscope placed therebetween. Stationary clasp of the barrel clamp 21 and moveable clasp of the barrel clamp 22, with the tensioner 20 may comprise one straightforward embodiment of the attachment of the device to the barrel of the laryngoscope, as also seen in FIG. 5, an isometric side view showing the details of the barrel clamp components from a low angle view. The combination of these clamps pass around the barrel 22 and thus combine to create a band in secure physical engagement to the barrel. The band may be metal, plastic, polymer, fabric, or combinations thereof.

The geometry of the attachment device may be keyed to the laryngoscope in order to prevent the device from being installed incorrectly, for example, upside down. In addition, the physical attachment of the accessory to the laryngoscope may be achieved through a frictional engagement or through a geometrical engagement.

Note that in embodiments, the two clasps 21, 22 may be replaced by metal bands, fabric straps, polymer straps or bands and so on, while the fastener device 20 may be replaced by a clamp, a clip, a snap, a buckle, a thumb-screw, hook-and-loop fabric, elastic and combinations thereof so as to secure the attachment to the barrel of a laryngoscope placed into the attachment. The attachment may also in embodiments be a screw or plate attached directly to the laryngoscope in a slightly more permanent manner, and in alternative embodiments, the entire device may in fact be a new type of laryngoscope, that is, rather than a retrofit, it is conceivable to provide the invention as original equipment.

In addition to the axis of the barrel of the laryngoscope, the barrel also has a circumference, in particular the distance around the barrel, normally a quantity chosen for convenience by medical personnel. Obviously, this same quantity defines the preferred embodiment's inner diameter of the attachment 29, thus providing a tight/secure physical engagement (locking in place, frictional engagement or other physical lock) between the barrel and the attachment 29. The attachment may engage a keying feature on the rail (against a corresponding feature on the laryngoscope) for proper alignment with the axis of the blade.

Lens rail 23 is secured to the attachment and thus is not normally movable in relation to the laryngoscope barrel. Lens slide 24 however, is disposed in operative physical relationship with lens rail 23 so that slide 24 may travel on rail 23 without coming loose therefrom. In the preferred embodiment, the motion of the slide 24 on rail 23 may be constrained by friction so that the slide does not move unless the user deliberately moves it, or the constraint may be cogs and traveling gears, notches, thumb-wheels and so on. In any case, the slide, and thus the progressive lens, may move parallel to the axis of the cylindrical barrel of the laryngoscope.

Pivot link 25 attaches the slide 24 to a lens frame 27 via pivot fasteners 26, thus allowing lens frame 27 to rotate in relation to the axis of the barrel of the laryngoscope, in turn allowing—progressive magnification lens 28 to be rotated as well.

It will be seen that a wide range of motion is thus available to the lens 28, including at least one dimension of translation (motion) and one dimension of rotation (pivoting), but in alternative embodiments, additional degrees/dimensions of motion, up to a complete 6 degrees of motion, may be provided. For the sake of simplicity, the preferred embodiment allows the two degrees of motion described; however, alternative embodiments may allow additional or alternative degrees of motion.

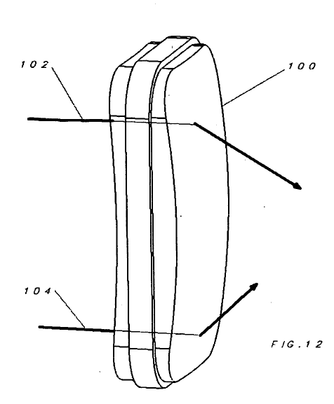

FIG. 12 is a cross-sectional view of a simple progressive lens having at least two different optical powers at different locations of the lens. Progressive lens 100 is not necessarily symmetrical between upper and lower portions, as shown by the bending of first light ray 102 versus the degree of bending of second light ray 104. The measurement in diopters of lens optical power is mathematically expressed as the inverse of the focal length of the lens, and it may be seen that the focal length of the upper part of the lens 100 is greater than that of the lower part of the lens, indicating greater optical power of the lower half.

While the term “progressive” lens is usually used to refer to a lens other than a bifocal lens, for the present patent application, the term progressive is taken to include bifocal and trifocal lenses and other multi-focal step-wise lenses having discontinuities of optical power, in addition to smoothly graduated lenses like that pictured. Note that a “true” progressive lens is much preferred over a bifocal arrangement having discontinuities in optical power, as the medical personnel using the device may easily adjust the optical power of the device (the inverse of the focal length of the lens) merely by adjusting it slightly up and down. Obviously a bifocal, trifocal, etc, arrangement rather than a true progressive lens may be used in alternative arrangements but as this does not provide to the user a smooth change in power, this is not presently the preferred embodiment, a true progressive lens is preferred. In addition, a bifocal arrangement or the like may (or may not) have a distracting line passing across it, which is not presently preferred.

A smooth range of optical power from roughly +5 to +8 diopters has been tested and found satisfactory for most personnel and purposes, however, the invention is not so limited. Optical powers from +1 to +12 diopters may be used, or even powers outside the ranges.

The lens may have, indicia thereon to aid in intubation, although this is an alternative embodiment of the invention.

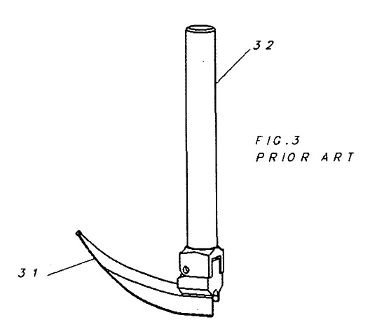

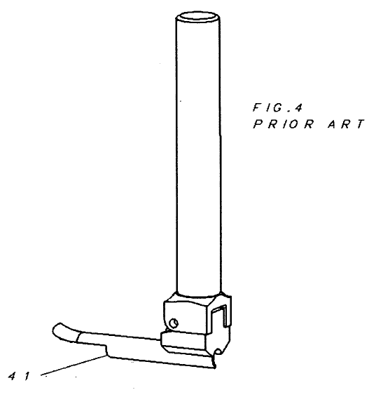

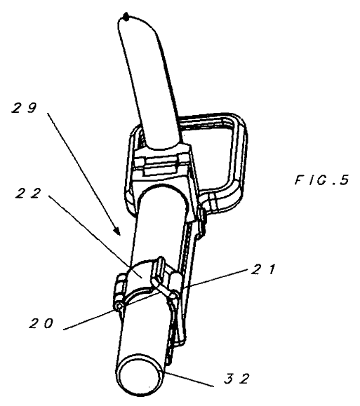

FIG. 3 is an isometric view of a typical curved-blade laryngoscope, while FIG. 4 is an isometric view of a typical straight-blade laryngoscope.

It should be readily understood that the device of the invention may easily be attached to and removed from a wide range of types of laryngoscopes, especially laryngoscopes having different circumferences of barrels, as the attachment 29 will easily fit to practically any laryngoscope barrel 32.

The device may also be used with laryngoscopes having different blades such as laryngoscope curved blade 31 or laryngoscope straight blade 41, or other types of blades, or with other types of equipment at the blade end, as the attachment to the barrel 32 means that the attachment is irrelevant to the type of blade. Because of the design of the present invention, has nothing to do with the fit of the invention to the laryngoscope: the device of the invention may be fitted regardless of type of blade.

The difference in blades is quite important. The laryngoscope and blade used for premature infant intubation (a premature baby may weigh less than one pound (under one half kilogram) and may be born in need of immediate breathing assistance) may be entirely different from the laryngoscope and blade used on a small adult, which may be different again from that used on an overweight individual or a child, and so on. Thus, it is not just commercially advantageous to provide a device which easily fits to the installed base of laryngoscopes already in use, it is also medically important to provide a device which may be used on the universe of available and in use laryngoscopes and may even be added at a moment's notice when the need for viewing assistance suddenly becomes apparent.

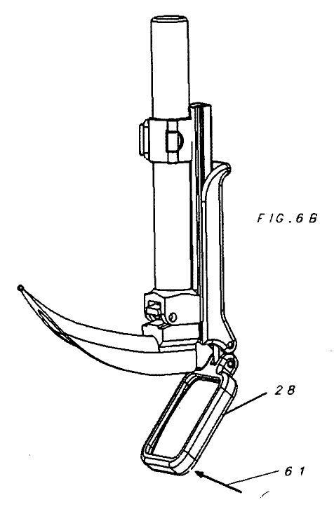

FIG. 6 and FIG. 6B are isometric demonstration diagrams showing articulation of the lens of the preferred embodiment of the invention. It may be clearly seen that the lens 28 may be pivoted in a first direction as shown by arrow 61 to achieve a first angular position in relation to the laryngoscope. Lens 28 may also be pivoted in a second direction as shown by arrow 62 to achieve a second angular position in relation to the laryngoscope, which advantage allows the device to be used in much more flexible manner and under a wider range of conditions.

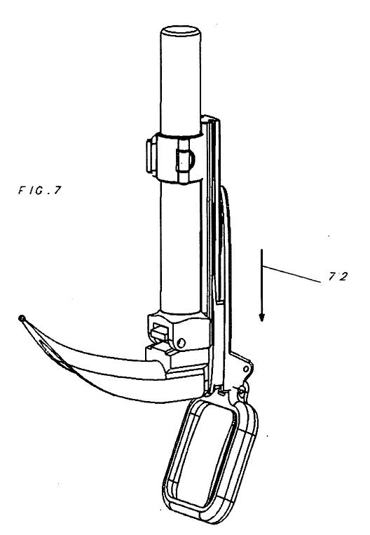

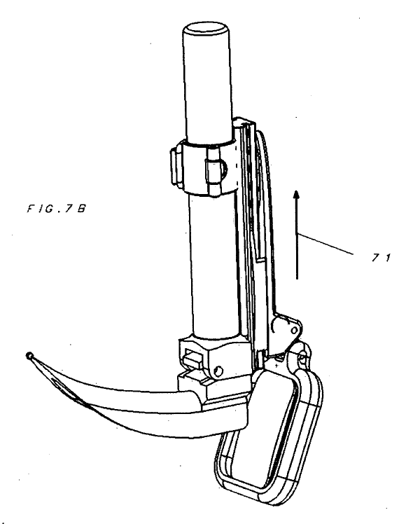

FIG. 7 and FIG. 7B are isometric side view which demonstrate longitudinal position adjustment of the lens guide. Arrow 71 indicates a first direction of translation (linear motion) while arrow 72 shows a second direction of translation (linear motion), both achieved by motion of the slide on the rail. As the slide moves, in carries the lens, lens frame and pivot mechanisms along with it.

It will be appreciated that the medical personnel using the device are operating under conditions of some degree or another of stress and with one or both hands already busy carrying out the intubation procedure. Under the circumstances of use, ease of use is a prerequisite and in fact, the preferred embodiment is preferred because a user holding the barrel of the laryngoscope in one hand may with the thumb of the same hand adjust the device in both rotation and translation, that is, in both angle of pivot and height along the rail. This one thumb and thus one handed use has been tested and found superior to schemes in which the user is forced to use two hands to adjust additional mechanisms.

It will be understood that by contrast, the prior art known by and large does not allow instantaneous and one-handed adjustment by stressed real-world medical personnel with their hands full, in particular, the practitioner most probably has the actual ET tube itself in the hand not holding the laryngoscope, and setting down the ET tube to adjust the laryngoscope may not be desirable.

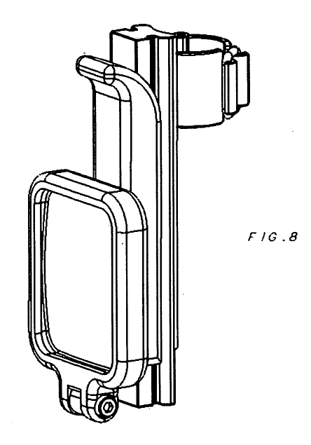

FIG. 8 is an isometric view of the preferred embodiment of the invention with the lens frame in the folded or parked position. During times of use, medical personnel will decide that the lens is entirely un-necessary, and yet will not have to engage in any two-handed activities or equipment juggling: the device may be simply “thumbed” out of the way in either or both rotation and translation and the medical person may continue her or his work without any significant interruption. This in turn makes it easier for medical personnel to simply leave the device attached as a matter of course, meaning that the odds of the device being available in the real-world on no notice will be dramatically increased.

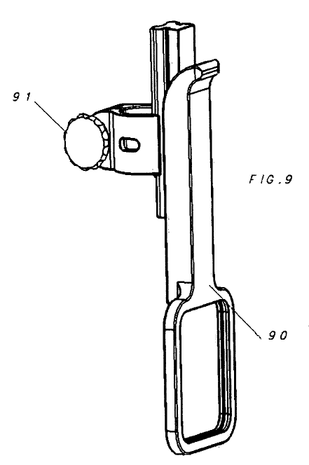

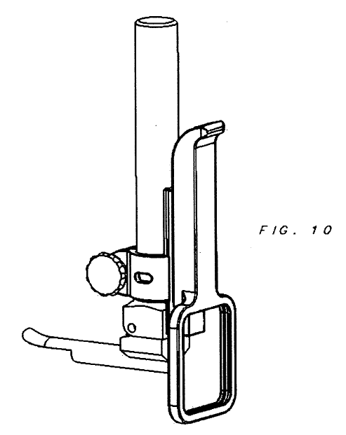

FIG. 9 is an isometric view of an alternative embodiment of the invention. This embodiment obeys the one-thumb principle by means of a thumb screw 91 which may be used to fasten the attachment to the barrel of the laryngoscope and in yet other alternative embodiments, may even by used to adjust the height of the device along the rail. Integrated lens frame and lens slide lack a pivot in this alternative embodiment, demonstrating that such pivoting action may be dispensed with for certain embodiments. FIG. 10 is an isometric view of this alternative embodiment of the invention attached to a straight-blade laryngoscope. Note that since linear motion of the device on the rail is permitted, the differing degrees of optical power available in the progressive lens may well be accessed without any other adjustment, and further without the practitioner being required to move her or his head into a different location. This freedom from head movement is important as the small airway and throat and mouth of a small human being does not actually allow a great deal of choice of head location: the practitioner's head must be able to see straight or virtually straight down the throat, and should be positioned quite near to the throat. In use, the practitioner may easily adjust the angle and location of the progressive lens.

Before and after use, the accessory of the invention may be easily added to or removed from a laryngoscope. To add the accessory to the laryngoscope, the practitioner will pass the barrel of the laryngoscope into the attachment, locate a keying feature on the rail against a corresponding feature on the laryngoscope for proper alignment with the axis of the blade, and tighten the attachment to secure it to the laryngoscope barrel. The clamp style device shown in FIG. 5, for example, will be positioned so that the short segment of the rail butts against the wide end of the laryngoscope barrel, automatically locating the lens in the general vicinity of the laryngoscope aiming channel. Once the magnification attachment is properly positioned on the laryngoscope barrel, the user performs the intubation procedure as usual, using the thumb of the laryngoscope gripping hand to slide the lens to the most effective position for clear viewing of the vocal cords. At the conclusion of the intubation procedure the attachment may be easily removed from the laryngoscope simply by loosening the fastener (thumbscrew, O-ring, etc) and then sliding the device off the barrel of the laryngoscope, followed by cleaning and storage for future use. Alternatively, the lens may simply be pivoted out of the way and the attachment left in place for future use.

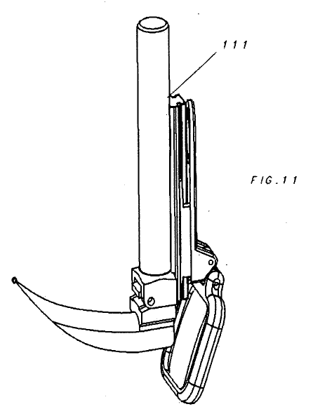

FIG. 11 is an isometric view of an alternative embodiment of the invention integral to a straight-blade laryngoscope. In this embodiment, the device of the invention is integrated as original equipment with the laryngoscope, barrel/rail assembly 111 may be a single piece or may be affixed together at the time of manufacture. Sub-embodiments of this embodiment may have the optical equipment either detachable or permanently affixed. This alternative embodiment is simpler, however, it may require the practitioner to “re-learn” on a new laryngoscope, and thus is not presently the preferred embodiment.

All components of the device are manufactured from autoclave compatible metal or polymer materials or combinations thereof.

The disclosure is provided to allow practice of the invention by those skilled in the art without undue experimentation, including the best mode presently contemplated and the presently preferred embodiment. Nothing in this disclosure is to be taken to limit the scope of the invention, which is susceptible to numerous alterations, equivalents and substitutions without departing from the scope and spirit of the invention. The scope of the invention is to be understood from the appended claims.

What is claimed is:

1. A laryngoscope accessory for use with a laryngoscope having a barrel having a circumference, the accessory comprising:
an attachment dimensioned and configured to engage such barrel of such laryngoscope, and:
a progressive lens secured to the attachment, the progressive lens having at least first and second different optical power ratings.

2. The laryngoscope accessory of claim 1, wherein the attachment further comprises:
at least one circumferential member passing about such circumference of such barrel, and:
at least one fastener secured to the circumferential member so as to force the circumferential member into secure physical engagement with such barrel.

3. The laryngoscope accessory of claim 2, wherein the circumferential member further comprises:
a band.

4. The laryngoscope accessory of claim 2, wherein the circumferential member further comprises:
a strap.

5. The laryngoscope accessory of claim 1, wherein the progressive lens further comprises:
a graduated progressive lens having no discontinuities of optical power.

6. The laryngoscope accessory of claim 1, wherein the progressive lens further comprises:
a bifocal lens.

7. The laryngoscope accessory of claim 1, wherein the progressive lens further comprises:
a trifocal lens.

8. The laryngoscope accessory of claim 1, wherein the progressive lens further comprises:
a lens frame secured to at least one portion of the progressive lens.

9. The laryngoscope accessory of claim 8, wherein the lens frame further comprises:
a pivot allowing the angle of the progressive lens to be adjusted relative to such blade of such laryngoscope.

10. The laryngoscope accessory of claim 1, wherein the attachment further comprises:
a rail affixed to the attachment and thereby fixed relative to such laryngoscope, and:
a slide movably disposed upon the rail, the progressive lens movably attached to the attachment via the slide, whereby:
the progressive lens may be moved parallel to such axis of such barrel of such laryngoscope.

11. The laryngoscope accessory of claim 1, wherein the fastener further comprises one member selected from the group consisting of: a clamp, a clip, a snap, a buckle, a thumb-screw, hook-and-loop fabric, elastic and combinations thereof.

12. A laryngoscope comprising:
a cylindrical barrel;
a blade attached to a first end of the barrel and extending, curved, therefrom;
a progressive lens secured to the barrel near the first end, the progressive lens having at least first and second different optical power ratings.

13. The laryngoscope of claim 12, wherein the progressive lens further comprises:
a graduated progressive lens having no discontinuities of optical power.

14. The laryngoscope of claim 12, wherein the progressive lens further comprises:
a bifocal lens.

15. The laryngoscope of claim 12, wherein the progressive lens further comprises:
a multifocal step-wise lens.

16. The laryngoscope of claim 12, wherein the progressive lens further comprises:
a lens frame secured to at least one portion of the progressive lens.

17. The laryngoscope of claim 16, wherein the lens frame further comprises:
a pivot allowing the angle of the progressive lens to be adjusted relative to the blade of the laryngoscope.

18. The laryngoscope of claim 12, further comprising:
a rail affixed to the barrel, and:
a slide movably disposed upon the rail, the progressive lens movably attached to the laryngoscope via the slide, whereby:
the progressive lens may be moved parallel to such axis of the barrel of the laryngoscope.
